(19) United States
(12) Patent Application Publication
(10) Pub. No.: US 2017/0202569 A1
(43) Pub. Date: Jul. 20, 2017

(54) Laparoscopic Surgical Device and Related Systems and Methods

(71) Applicant: Miret Surgical, Inc., Elmhurst, IL (US)

(72) Inventors: John Avi Roop, Scottsdale, AZ (US); Christopher Pell, San Francisco, CA (US)

(21) Appl. No.: 15/475,766

(22) Filed: Mar. 31, 2017

(51) Int. Cl. A61B 17/28

(52) U.S. Cl. CPC A61B 17/282; A61B 2017/00473

(57) ABSTRACT

The various embodiments herein relate to surgical systems having a surgical tool, a detachable tool head, and an introducer tool. The various systems can be used for minimally invasive laparoscopic surgical procedures.

### FIELD OF THE INVENTION

The various embodiments herein relate generally to apparatus, systems, and methods for performing surgery, e.g., laparoscopic surgery, and more particularly, to laparoscopic surgical tools and to systems and methods including such tools.

### BACKGROUND OF THE INVENTION

Surgery has become increasingly less invasive thanks to advances in medical technology. Laparoscopy is the dominant minimally invasive surgical (MIS) approach used today and has replaced many traditional “open” approaches. In laparoscopic surgery, trocars (typically 3-5) are placed at separate points in the surgical field. These trocars serve as ports into a body cavity or other surgical space (such as the abdomen) through which special long and thin instruments can be inserted. Manipulation of these tools from outside the body mechanically translates into motion within the body cavity. Depending on the tool head design, different instruments have different functions. The appropriate instrument is selected based on what the surgeon needs for that step of the procedure.

Minimally Invasive Surgery (MIS) offers several advantages compared to open surgical procedures including minimal trauma to the abdominal wall and hence less postoperative pain, fewer wound complications, earlier patient mobilization, and shorter length of stay. Laparoscopic access to the peritoneal space is the dominant MIS approach when performing minimally invasive abdominal operations.

Recent clinical studies show that further reduction of the size and/or number of incisions may offer added benefits such as faster recovery, less pain, reduced operative time, and improved cosmetic result. Such benefits may have physical and psychological impact. However, the size of the tool tips on conventional instruments used in laparoscopic procedures generally limit the ability to reduce the size of the incisions and trocars needed for such procedures.

Recent waves of scar-free techniques, including natural orifice trans-luminal endoscopic surgery (“NOTES”) and single-port surgery, have emerged to meet the need to further reduce the incisions required for surgical procedures. Ample information explaining the details of these new approaches exists in the public domain. Of the two, single-port surgery is thought among the surgery community to be the more feasible approach given available technology today.

Single-port surgery involves a multi-channel port that is typically placed in the belly button. This results in hidden scar post operatively. Through these channels, standard laparoscopic tools can be inserted. However, manipulation is more challenging because the tight aperture of the belly button and strong connective tissue in the abdominal wall forces all the instruments to move dependent on one another. In addition, the surgeon's hands are crowded together because of these constraints. Triangulation is largely lost. This makes the procedure frustrating to perform compared to other approaches.

A number of commercially available tools have been designed to circumvent some of these limitations. Some are variations of standard laparoscopic instruments but have articulating tool heads. Such designs are intended for re-enabling triangulation. However, constraints of the belly button port may force these articulating tools to cross, thus reversing the left-right motion between what the surgeon does with his hands and what he sees on the video monitor. Also, the complex mechanics behind the articulation may drive the cost up significantly.

The need exists for a revised laparoscopic technique and tools that reduce surgery-induced trauma but preserve the ergonomics and visualization to which surgeons have become accustomed. Such a procedure and tools may be safer for patients. A scar-free or minimal scar result may also appeal more to young adults, and the potential health benefits of a less traumatic approach may be higher for children and the elderly.

The first step during a laparoscopic surgical procedure is to insufflate the body cavity with a harmless gas (such as carbon dioxide) to increase the working space for the tools. The trocars are inserted across the abdominal wall and are designed to prevent excessive leakage of the insufflation gas, which invariably happens with incisions greater than three millimeters (3 mm).

In endoscopic and laparoscopic surgical procedures, a trocar device is used to puncture the patient's body in order to provide an access port through the abdominal wall to allow for the introduction of surgical instruments. A typical trocar requires a one-centimeter incision. Typically, a first trocar is placed above the umbilicus to introduce a camera to allow the surgeons to view the surgical site. The camera view is projected on a screen outside the body, which the surgeon and his or her assistants watch in order to appropriately manipulate the instruments inside the body cavity. Additional trocars are used to introduce surgical instruments, such as grasping tools, scissors, clips, and electrosurgical instruments. Typically, the laparoscopic instruments extend toward the surgical target from either side of the video camera. This “triangulation” of the instruments provides the most ergonomic and intuitive set up for the surgeon.

Patients who undergo laparoscopic surgery may benefit from shorter hospital stays and reduced surgery-inflicted morbidity compared to those who undergo open surgery. But, the number of trocar ports used in an operation is trauma-limited. For many cases, surgeries requiring more than five to seven (5-7) ports may be better performed using an open approach. Surgeons often hesitate to place more ports, even if it would mean making the procedure easier to do, because of the increased risk of wound complications with each additional incision (such as infection, dehiscence, or hernia).

There is a need in the art for improved laparoscopic surgical devices.

### BRIEF SUMMARY OF THE INVENTION

Discussed herein are various laparoscopic surgical tools and related systems and methods, all designed to facilitate surgery while minimizing the number and/or size of surgical access sites used and/or minimizing visible scars related thereto.

In Example 1, a surgical system comprises a surgical tool comprising a handle and a control shaft, a detachable tool head operably coupleable to the control shaft, and an introducer tool comprising an open coupler. The open coupler comprises first and second jaws, a seat defined by inner walls of the first and second jaws, and an opening defined by a distal end of the first and second jaws.

Example 2 relates to the surgical system according to Example 1, wherein the detachable tool head comprises a tool head shaft and a tool head end effector operably coupled to the shaft, wherein the tool head end effector has a diameter greater than a diameter of the introducer tool.

Example 3 relates to the surgical system according to Example 2, wherein the detachable tool head is operably coupleable to the open coupler of the introducer tool.

Example 4 relates to the surgical system according to Example 3, wherein the tool head shaft is positionable within the seat of the open coupler via the opening, thereby resulting in the coupling of the detachable tool head to the open coupler.

Example 5 relates to the surgical system according to Example 3, wherein the detachable tool head is operably coupleable to the open coupler prior to introduction of the open coupler into a target cavity of a patient.

Example 6 relates to the surgical system according to Example 5, wherein the detachable tool head is operably coupleable to the open coupler prior to introduction of the open coupler into the target cavity via an incision or a port.

Example 7 relates to the surgical system according to Example 5, wherein the control shaft is positionable into the target cavity.

Example 8 relates to the surgical system according to Example 7, wherein the control shaft is configured to be coupled to the detachable tool head within the target cavity.

Example 9 relates to the surgical system according to Example 8, wherein the introducer tool is configured to be uncoupled from the detachable tool head and retracted from the target cavity.

In Example 10, a surgical system for assembly within the thoracic cavity comprises an introducer tool configured to inserted into the thoracic cavity via an incision or port, a detachable tool head configured to be operably coupled to the introducer tool prior to insertion into the thoracic cavity, and a surgical tool configured to be introduced into the thoracic cavity separately from the introducer tool. The introducer tool comprises an open coupler comprising first and second jaws, a seat defined by inner walls of the first and second jaws, and an opening defined by a distal end of the first and second jaws. The detachable tool head comprises a lung grasping end effector, wherein the end effector has a diameter greater than the introducer tool. The surgical tool comprises a handle and a control shaft, wherein the surgical tool is configured to be coupled to the detachable tool head within the thoracic cavity.

Example 11 relates to the surgical system according to Example 10, wherein the introducer tool is configured to be uncoupled from the detachable tool head after the surgical tool is coupled to the detachable tool head.

Example 12 relates to the surgical system according to Example 11, wherein the lung grasping end effector is configured to manipulate a lung within the thoracic cavity after the surgical tool is coupled to the detachable tool head.

Example 13 relates to the surgical system according to Example 11, wherein the introducer tool is configured to be removed from the thoracic cavity after the introducer tool is uncoupled from the detachable tool head.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1A is a side view of a surgical tool including a handle and control shaft with a modular tool head coupled to the shaft, showing an end effector of the tool head in the open position, according to one embodiment.

FIG. 1B is a close-up perspective view of the tool head of FIG. 1A, showing relative alignment of internal features of the tool head and control shaft.

FIG. 1C is a side view of the surgical tool of FIG. 1A, showing the end effector of the tool head in the fully closed position, according to one embodiment.

FIG. 1D is a close-up perspective view of the tool head of FIG. 1C, showing relative alignment of internal features of the tool head and control shaft.

FIG. 1E is a side view of the surgical tool of FIG. 1A with an actuator on the handle directed to an alignment position in which the tool head may be coupled or removed from the shaft.

FIG. 1F is a close-up perspective view of the tool head of FIG. 1E, showing relative alignment of internal features of the tool head and control shaft.

FIGS. 2A and 2B are top and side views, respectively, of an exemplary embodiment of an outer cannula tube that may included in the shaft of the surgical tool of FIGS. 1A, 1C, and 1E.

FIGS. 3A and 3B are top and side views, respectively, of an exemplary embodiment of an inner “active” shaft tube that may included in the shaft of the surgical tool of FIGS. 1A, 1C, and 1E.

FIG. 4A is a top view of a tool carrier, according to one embodiment.

FIG. 4B is a side view of the tool carrier of FIG. 4A.

FIG. 4C is a perspective view of the tool carrier of FIG. 4A.

FIG. 4D is a close-up top view of the coupler of the tool carrier of FIG. 4A.

FIG. 5A is a perspective view of a tool head, according to one embodiment.

FIG. 5B is an exploded perspective view of the tool head of FIG. 5A.

FIG. 6A is a top view of a tool head, according to another embodiment.

FIG. 6B is a top view of another tool head, according to a further embodiment.

FIG. 7 is a perspective view of a tool head coupled to a carrier, according to one embodiment.

FIG. 8 is a perspective view of a carrier disposed through an incision, according to one embodiment.

FIG. 9 is a perspective view of a shaft of a surgical tool being positioned adjacent to a tool head coupled to a carrier prior to assembly, according to one embodiment.

### DETAILED DESCRIPTION

The various embodiments disclosed or contemplated herein are directed to devices, systems, and methods for performing surgery, e.g., laparoscopic surgery. More particularly, provided herein are laparoscopic surgical tools and to systems and methods including such tools, e.g., designed to facilitate surgery while minimizing the number and/or size of access sites used and/or minimizing visible scars.

For example, in one embodiment, a modular surgical instrument may be provided that enables standard laparoscopic techniques to be performed through relatively small puncture holes in the body wall of a patient used to access the surgical space. In an exemplary embodiment, the assembled modular instrument may include a handle, a relatively small diameter cannular shaft (e.g., less than or equal to about 2.5 millimeter diameter), and one or more interchangeable tool heads. The cannular shaft may include two coaxial shafts that move relative to one another, e.g., to actuate a tool head attached to the shaft in situ.

The tool head may be introduced into the surgical site independently of the cannular shaft, e.g., through separate trocar ports, punctures, or other access passages, e.g., located through the umbilicus and the like. For example, the tool head can be introduced using a tool carrier. The cannular shaft may be attached to the tool head within the surgical space inside the body. Once the modular instrument is fully assembled, the tool head may be manipulated through the puncture hole, e.g., using an actuator on the handle, to perform one or more procedures within the surgical space.

In an exemplary embodiment, a coaxial locking mechanism is provided between the cannular shaft and the tool head, e.g., that locks both an external shaft and an internal “active” shaft on the cannular shaft with respective elements on the tool head, e.g., as disclosed in International Publication No. WO 2010/114634, which is hereby incorporated herein by reference.

In accordance with another embodiment, an apparatus (also referred to herein as a “tool carrier”) is provided for delivering a tool head into a surgical space within a patient's body being visualized by an endoscope introduced into the surgical space via an access port. Generally, the apparatus includes an elongate shaft including a proximal end and a distal end, the shaft connectable to a shaft of an endoscope such that the shaft is advanceable from a proximal position to a distal position, e.g., such that the distal end is within a field of view of the endoscope. An end effector may be provided on the distal end of the tool carrier for releasably engaging a tool head, and a handle may be provided on the proximal end of the shaft including an actuator for operating the end effector to engage and release a tool head.

In accordance with still another embodiment, a system or kit is provided for performing a procedure within a surgical space within a patient's body that includes a plurality of tool heads, and a tool head carrier including features for removably receiving one or more tool heads, the tool head carrier sized for introduction through a trocar or other port into a surgical space. The system or kit may also include a tool shaft introduceable into the surgical space and include features for securing a tool head to the tool. Each tool head may include a proximal end including a connector for coupling the tool head to a distal end of a tool shaft, and a distal end including an end effector for performing a surgical procedure.

In accordance with a further embodiment, a method is provided for performing a procedure within a surgical space within a patient's body that includes introducing a tool head into a surgical space through a trocar, other port, or an incision, and securing the tool head to a tool shaft for performing a procedure within the surgical space. The tool head may be introduced into the surgical space on a tool head carrier, which may carry one or more tool heads, e.g., along an endoscope.

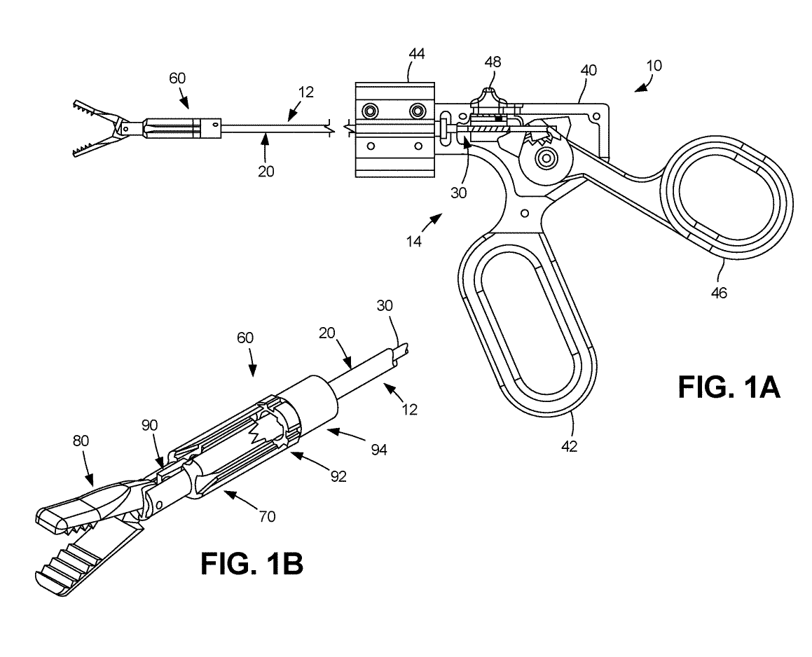

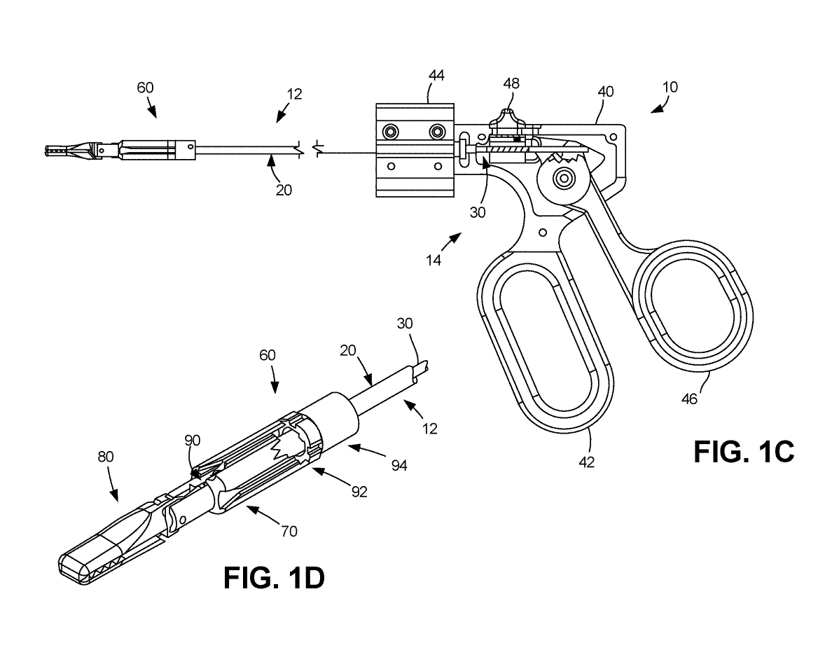

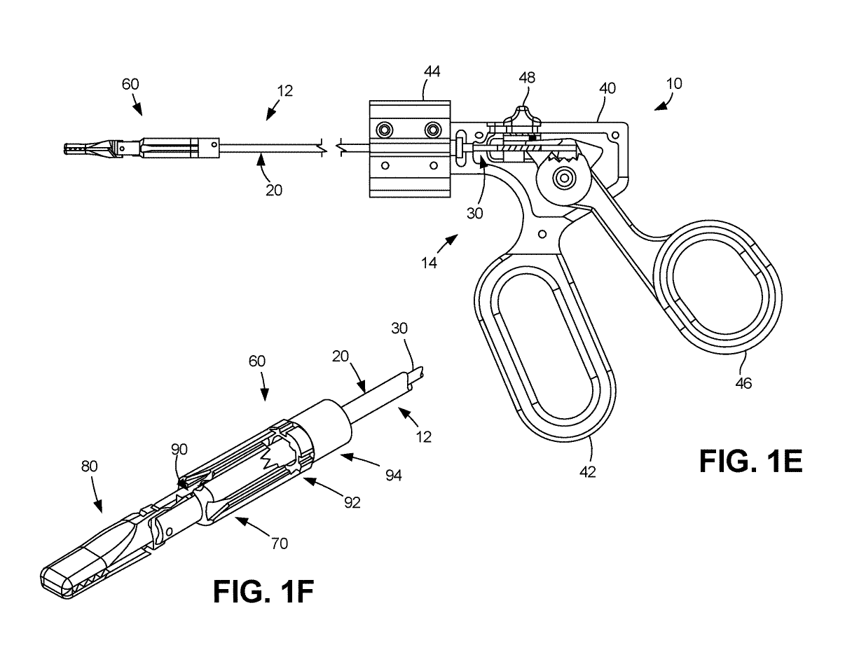

Turning to FIGS. 1A-1F, an exemplary embodiment of a surgical tool 10 is shown that includes a control shaft 12 and a handle 14, and has a modular tool head 60 coupled to the control shaft 12. Generally, similar to other embodiments described herein and in the references incorporated by reference herein, the control shaft 12 includes an outer cannular tube or shaft 20 and an inner “active” shaft 30 that are movable relative to one another, e.g., axially and/or rotationally, as described elsewhere herein. In addition, the outer and inner shafts 20, 30 include features, e.g., on distal ends 22, 32 as shown in FIGS. 2A-3B, for engaging corresponding features on the tool head 60, e.g., to couple the tool head 60 to the control shaft 12 and/or actuate an end effector 80 on the tool head 60, also as described further below.

For example, as shown in FIGS. 2A and 2B, the distal end 22 of the outer cannular shaft 20 includes a longitudinal slot 24, e.g., extending proximally from a distal tip 23 of the cannular shaft 20 to an angular pocket 26 that extends laterally from the slot 24 to provide a blunt distal edge 26a. As shown in FIGS. 3A and 3B, the inner shaft 30 also includes a longitudinal slot 34, e.g., extending proximally from a distal tip 33 of the inner shaft 30. An angular notch 36 is provided in the distal end 32 at an intermediate location along the slot 34. Initially, the slots 24, 34 may be aligned with one another, e.g., to accommodate receiving the tool head 60 onto the control shaft 12, yet may be directed out of alignment with one another, e.g., by rotating the inner shaft 30 relative to the outer shaft 20, to secure the tool head 60 to the control shaft 12.

The distal tips 23, 33 may be generally coextensive with one another, although the distal tip 33 of the inner shaft 30 may extend from or retract into the distal end 22 of the outer cannular shaft 20, e.g., when the inner shaft 30 is directed axially relative to the outer cannular shaft 20. As shown, the distal tips 23, 33 are beveled, e.g., sharpened to facilitate penetration through tissue, if desired, and/or to facilitate insertion into the tool head 60. Alternatively, the distal tips 23, 33 may be blunt or have other configurations, as desired.

Returning to FIGS. 1A-1F, the handle 14 includes a stationary housing 40 from which a hand rest 42 extends, e.g., configured to be held by a user. The outer cannular shaft 20 may be substantially fixed axially and rotationally relative to the housing 40. The inner shaft 30 may be rotated relative to the housing 40, for example, using actuator 44, e.g., to rotate the inner shaft 30 relative to the cannular shaft 20 to lock or unlock the tool head 60 from the control shaft 12. Alternatively, if desired, the actuator 44 may be coupled to the outer cannular shaft 20, which may be rotatable to lock or unlock the tool head 60 from the control shaft 12.

The handle 14 also includes a trigger or other actuator 46 coupled to the inner shaft 30, e.g., for directing the inner shaft 30 axially relative to the outer cannular shaft 20. For example, with the tool head 60 coupled to the control shaft 12, the trigger 46 may be activated to direct the inner shaft 30 axially to open and close an end effector 80 of the tool head 60, as shown in FIGS. 1A and 1C.

Optionally, as shown, the handle 14 may include an alignment actuator 48 for directing the inner shaft 30 between first and second predetermined axial locations, e.g., with the trigger 46 in the closed position. For example, with the trigger 46 and the end effector 80 in the closed position shown in FIG. 1C, the alignment actuator 48 may directed from an operational position (shown in FIG. 1C) to a locked position in which components of the tool head 60 and control shaft 12 are aligned to allow the tool head 60 to be separated from (or coupled to) the control shaft 12.

The tool head or end effector assembly 60, according to one embodiment, may be selectively coupled to the shaft 12 of the surgical tool 10 of FIGS. 1A-1F. The tool head 60 includes a housing 70, an end effector 80, an actuating link 90 movable axially relative to the housing 70 and coupled to the end effector 80, a locking ring 92, and a guide cap 94.

Additional embodiments of surgical tools, including handles and tool heads, related methods, and additional details thereof, are described in further detail in U.S. Published Application 2013/0331646, entitled “Apparatus, Systems, and Methods for Performing Laparoscopic Surgery,” and also in U.S. Published Application 2016/0015253, entitled “Surgical Devices, Systems, and Methods,” both of which are hereby incorporated herein by reference in their entireties.

According to certain embodiments as discussed in further detail below, the tool head 60 can be introduced into a surgical space using a tool head carrier (such as tool head carrier 200 discussed below) and attached to the cannular shaft 20 for performing a surgical procedure within the surgical space.

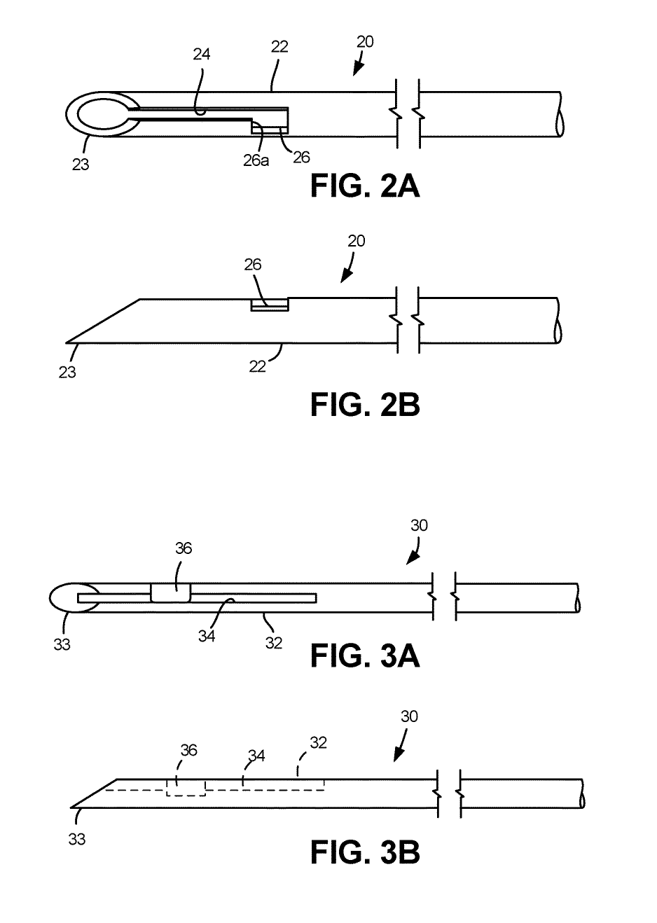

According to one embodiment as shown in FIGS. 4A-4D, the tool carrier 200 is an introducer tool 200 having an introducer body (also referred to as a “handle”) 202 and a coupler (also referred to herein as a “u-shaped coupler,” “open coupler,” “open-ended coupler,” “open-faced coupler,” “coupling jaws,” or “coupling component”) 204. The coupler 204 has two jaws 204A, 204B that define a seat 206 that is shaped to removably receive a tool head (such as tool head 60 discussed above, for example) such that the shaft of the tool head is disposed in the seat 206 and thereby coupled to the coupler 204. The seat 206 is defined by the inner walls of the jaws 204A, 204B, and is accessible by a shaft of a tool head (such as tool head 60) being moved through the opening 208 defined between the distal ends of the two jaws 204A, 204B and into the seat 206. The tool head can then be removed or uncoupled from the coupler 204 by moving the shaft out of the seat 206 through the opening 208.

In one embodiment, the open-ended coupler 204 of the tool carrier 200 (and any other tool carrier embodiment disclosed or contemplated herein) allows it to accommodate a tool head having jaws with a wider diameter than the diameter of the tool carrier 200, because the seat 206 is sized to accommodate the shaft of the tool head. The open-ended coupler 204 allows for coupling the tool head shaft to the coupler 204 and subsequently uncoupling the shaft from the coupler 204 via the opening 208 defined at the distal end of the jaws 204A, 204B as discussed above without having to move the jaws of the tool head (such as tool head 60) through the seat 206. As such, the open-ended coupler 204 can be used to introduce any tool head having jaws or other end effector components with a diameter greater than the tool carrier 200.

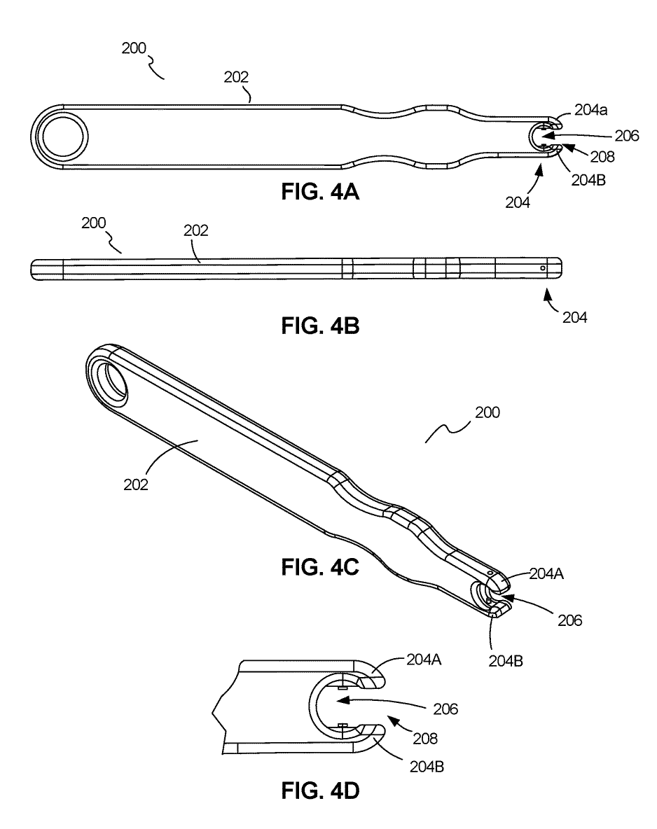

One such tool head 240 embodiment (with jaws having a diameter greater than that of the tool carrier 200) is provided for use with the tool carrier 200 (or any other system disclosed or incorporated by reference), as shown in FIGS. 5A and 5B. In certain implementations, this tool head 240 has a graspers end effector 244 configured for use in grasping and manipulating lung tissue. As an example, the tool head 240 may be selectively coupled to the shaft 12 of the surgical tool 10 of FIGS. 1A, 1C, and 1E. As shown in FIGS. 5A-5B, the tool head 240 includes a shaft (or “housing”) 242, an actuating link 246 movable axially relative to the housing 242 and coupled to the end effector 244, a locking ring 248, and a guide cap 250, generally similar to embodiments in the reference incorporated by reference herein. More specifically, it is understood that the actuating link 246, the locking ring 248, and the guide cap 250 operate in substantially the same way as described with respect to the tool head embodiments in U.S. Published Application 2013/0331646, which is incorporated above.

The end effector 244 includes a pair of grasper elements 244A, 244B. The first grasper element 244A has first and second ends 252A, 252B on opposite sides of an aperture 254 extending through the end effector element 244A. Similarly, the second grasper element 244B has first and second ends 256A, 256B on opposite sides of an aperture 258 extending through the element 244B. The two apertures 254, 258 combine—along with the aperture 260 on the housing 242—to provide a pivot axis 262, as best shown in FIG. 5A, via the pin 278 (as best shown in FIG. 5B) disposed therethrough. In the first grasper element 244A, the first end 252A includes a paddle-shaped jaw element 270 having a plurality of teeth or other gripping features 270A, while the second end 252B includes a tab 272 that may be slidably engaged with the actuating link 246, as described in further detail in the incorporated application. In the second grasper element 244B, the first end 256A includes a paddle-shaped jaw element 274 having a plurality of teeth or other gripping features 274A, while the second end 256B includes a tab 276 that may be slidably engaged with the actuating link 246, as described in further detail in the incorporated application. Both jaw elements 270, 274 have openings 280, 282 defined therethrough as shown.

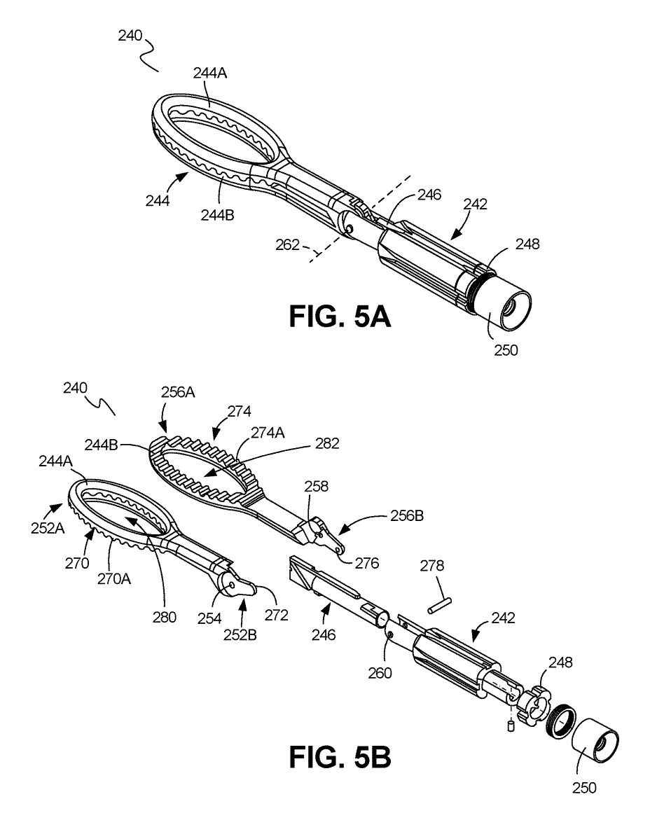

It will be appreciated that other cooperating elements may be provided instead of the grasper elements 244A, 244B on the tool head 240, e.g., curved and/or tapered elements (not shown) instead of the substantially flat elements shown, as described elsewhere in the reference incorporated by reference herein. Thus, according to certain alternative embodiments as best shown in FIG. 6A, the end effector 300 is a pair of grasper elements 302 having a substantially triangular shape. In a further alternative, as best shown in FIG. 6B, the end effector 310 is a pair of grasper elements 312 having a substantially triangular shape that is narrower than the pair of grasper elements 302 in FIG. 6A.

In accordance with one embodiment, one method of introducing tool head 240 (or any of tool heads 300, 310, for example, or any other tool head disclosed or contemplated herein) into a surgical space and attaching the tool head 240 to the cannular shaft 12 of surgical tool 10 (or any cannular shaft of any such surgical tool as disclosed or contemplated herein) for performing a surgical procedure within the surgical space can include the following steps.

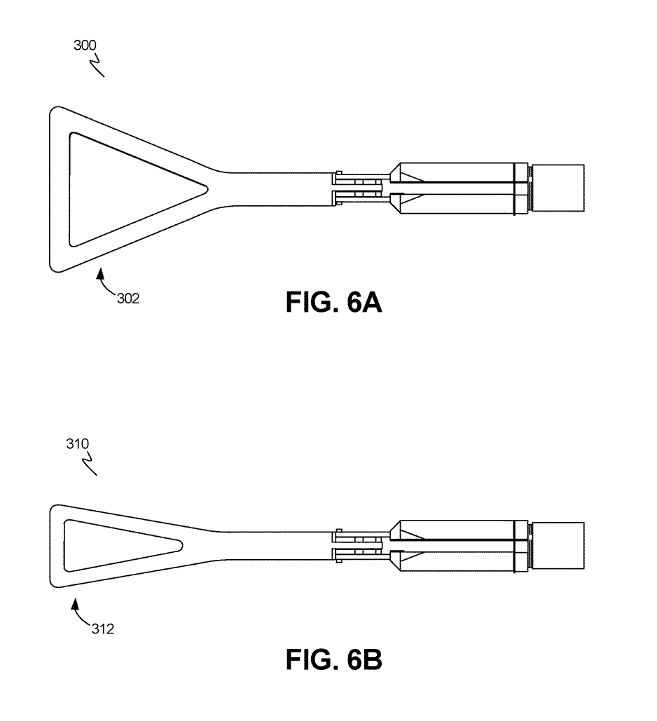

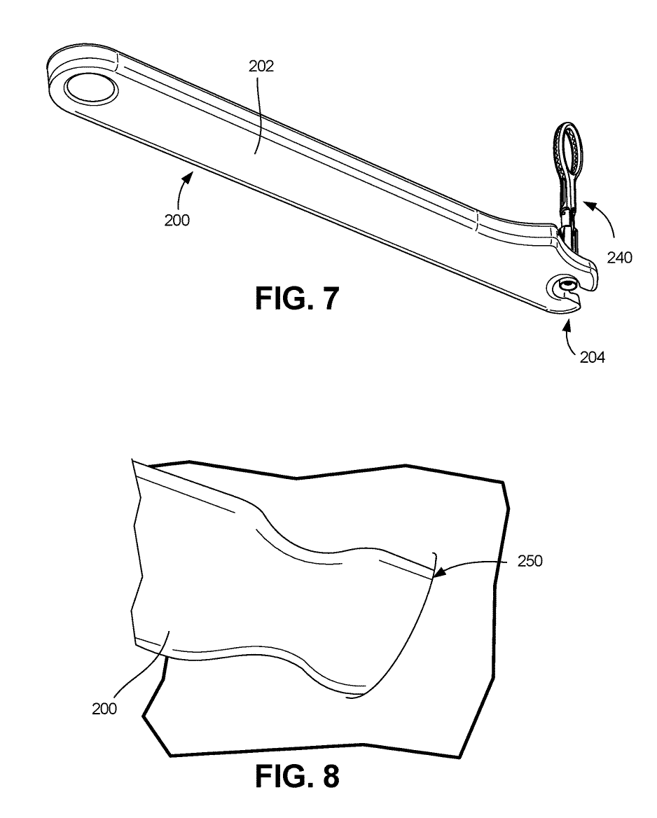

As shown in FIG. 7, the tool head 240 can be coupled or attached to the tool carrier 200 by inserting the tool head 240 into the coupler 204 via the opening 208 such that the shaft 242 of the tool head 240 is positioned in the seat 206 and thus coupled to the carrier 200. Once the tool head 240 is attached to the carrier 200, the carrier 200 (with the attached head 240) can be inserted into the body as shown in FIG. 8. More specifically, the carrier 200 with the tool head 240 is inserted through an incision 250 as shown such that the coupler 204 with the tool head 240 attached is disposed within the patient's body while the handle 202 of the carrier is disposed through the incision 250 such that a proximal end of the handle 202 extends out of the patient's body. Alternatively, the carrier 200 can be inserted through a port.

According to one implementation, at the same time as, prior to, or after the tool head 240 is introduced into the target surgical space of the patient with the carrier 200, the cannular shaft 12 of the surgical tool 10 can be inserted into the target cavity via an incision as well (or through a trocar or another port). In one embodiment, the incision through which the shaft 12 is inserted is different from the incision 250 through which the tool head 240 and carrier 200 are inserted.

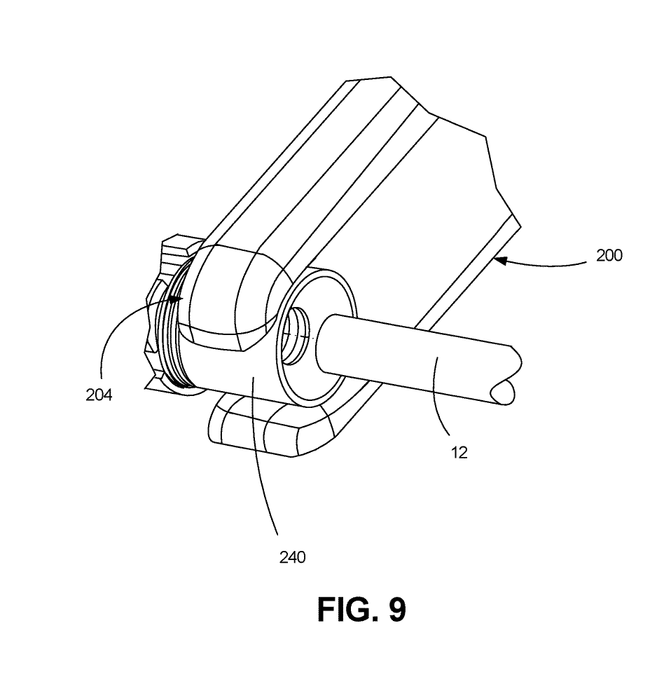

Once both the surgical tool shaft 12 and the carrier 200 with the tool head 240 are disposed in the patient's cavity, the carrier 200 and/or the shaft 12 can be positioned such that the tool head 240 is accessible for coupling with the distal end of the surgical tool shaft 12 as shown in FIG. 9, according to one embodiment. The shaft 12 is then inserted into the tool head 240, thereby coupling the tool head 240 to the shaft 12 (and thus the surgical tool 10). This coupling of the tool head 240 and the shaft 12 can be accomplished via camera visualization or direct visualization.

The carrier 200 is then uncoupled from the tool head 240 by simply pulling the handle 202 in a proximal direction such that the tool head 240 uncouples from the coupler 204 via the opening 208 in the coupler 204. The carrier 200 is then retracted from the body cavity via the incision 250 while the tool head 240 remains coupled to the surgical tool 10. At that point, the surgical tool 10 can be operated with the tool head 240 coupled thereto to perform the desired surgical procedure within the target cavity of the patient.

Optionally, another tool head may be introduced and coupled to the cannular shaft 12 in a similar manner. Thus, one or more tool heads may be introduced sequentially into a surgical space to perform a series of steps or procedures, as desired. Once the desired procedure(s) is completed, the carrier tool 200, cannular shaft 12, any endoscope (not shown) used in the procedure, and/or any other devices may be removed from the patient's body using conventional methods.

According to one implementation, the various embodiments disclosed or contemplated herein can be used to perform a thoracic surgical procedure across the rib cage of a patient. In certain of these implementations, the tool head 240 can be introduced via the carrier 200 to be coupled to the shaft 12 inside the thoracic cavity and used for the procedure, which includes grasping and manipulating lung tissue.

Although the present invention has been described with reference to preferred embodiments, persons skilled in the art will recognize that changes may be made in form and detail without departing from the spirit and scope of the invention.

What is claimed is:

1. A surgical system comprising:
(a) a surgical tool comprising a handle and a control shaft;
(b) a detachable tool head operably coupleable to the control shaft; and
(c) an introducer tool comprising an open coupler comprising:
(i) first and second jaws;
(ii) a seat defined by inner walls of the first and second jaws; and
(iii) an opening defined by a distal end of the first and second jaws.

2. The surgical system of claim 1, wherein the detachable tool head comprises:
(a) a tool head shaft; and
(b) a tool head end effector operably coupled to the shaft, wherein the tool head end effector has a diameter greater than a diameter of the introducer tool.

3. The surgical system of claim 2, wherein the detachable tool head is operably coupleable to the open coupler of the introducer tool.

4. The surgical system of claim 3, wherein the tool head shaft is positionable within the seat of the open coupler via the opening, thereby resulting in the coupling of the detachable tool head to the open coupler.

5. The surgical system of claim 3, wherein the detachable tool head is operably coupleable to the open coupler prior to introduction of the open coupler into a target cavity of a patient.

6. The surgical system of claim 5, wherein the detachable tool head is operably coupleable to the open coupler prior to introduction of the open coupler into the target cavity via an incision or a port.

7. The surgical system of claim 5, wherein the control shaft is positionable into the target cavity.

8. The surgical system of claim 7, wherein the control shaft is configured to be coupled to the detachable tool head within the target cavity.

9. The surgical system of claim 8, wherein the introducer tool is configured to be uncoupled from the detachable tool head and retracted from the target cavity.

10. A surgical system for assembly within the thoracic cavity, the system comprising:
(a) an introducer tool configured to inserted into the thoracic cavity via an incision or port, the introducer tool comprising an open coupler comprising:
(i) first and second jaws;
(ii) a seat defined by inner walls of the first and second jaws; and
(iii) an opening defined by a distal end of the first and second jaws;
(b) a detachable tool head configured to be operably coupled to the introducer tool prior to insertion into the thoracic cavity, the detachable tool head comprising a lung grasping end effector, wherein the end effector has a diameter greater than the introducer tool; and
(c) a surgical tool configured to be introduced into the thoracic cavity separately from the introducer tool, the surgical tool comprising a handle and a control shaft, wherein the surgical tool is configured to be coupled to the detachable tool head within the thoracic cavity.

11. The surgical system of claim 10, wherein the introducer tool is configured to be uncoupled from the detachable tool head after the surgical tool is coupled to the detachable tool head.

12. The surgical system of claim 11, wherein the lung grasping end effector is configured to manipulate a lung within the thoracic cavity after the surgical tool is coupled to the detachable tool head.

13. The surgical system of claim 11, wherein the introducer tool is configured to be removed from the thoracic cavity after the introducer tool is uncoupled from the detachable tool head.
